(19) United States
(12) Patent Application Publication
(10) Pub. No.: US 2017/0189317 A1
(43) Pub. Date: Jul. 6, 2017

(54) COMPOSITIONS AND METHODS FOR IMPROVING THE APPEARANCE OF THE SKIN

(71) Applicant: L'OREAL, Paris (FR)

(72) Inventors: Anne-Laure Suzanne BERNARD, New York, NY (US); Alexandra Jane Elisa FARRAN, Dayton, NJ (US)

(21) Appl. No.: 15/087,066

(22) Filed: Mar. 31, 2016

Related U.S. Application Data
(60) Provisional application No. 62/274,071, filed on Dec. 31, 2015.

(51) Int. Cl. A61K 8/895; A61K 8/19; A61K 8/25; A61K 8/04; A61Q 19/08; A61K 8/891

(52) U.S. Cl. CPC A61K 8/895; A61Q 19/08; A61K 8/891; A61K 8/25; A61K 8/042; A61K 8/19; A61K 2800/95; A61K 2800/594; A61K 2800/884

(57) ABSTRACT

The disclosure relates to compositions and methods for improving the appearance of the skin. Compositions comprise comprising (A) (a) at least one vinyl-terminated polysiloxane and at least one hydride-functionalized polysiloxane, and (b) at least one filler; and (B) (a) at least one vinyl-terminated polysiloxane; (b) at least one filler, and (c) at least one metal catalyst. Methods comprise applying the compositions to the skin to tighten the skin or hide skin imperfections by forming a film on the skin. Kits comprising the composition are also disclosed.

This application claims priority to U.S. Provisional Patent Application No. 62/274,071, filed Dec. 31, 2015.

### TECHNICAL FIELD

The disclosure relates to compositions and methods for improving the appearance of the skin.

### BACKGROUND

Skin is primarily comprised of two layers. The outer layer, or epidermis, has a depth of approximately 100 μm. The inner layer, or dermis, has a depth of approximately 3000 μm from the outer surface of the skin and is comprised of a network of fibrous protein known as collagen, which provides skin firmness, and elastin, which supplies skin elasticity and rebound. As a person ages, their skin produces less collagen and elastin each year. As a result, the skin becomes thinner and more fragile with age, and wrinkle formation as a result of aging is inevitable.

In addition, as a person ages, other skin imperfections may appear or become more noticeable. For example, age spots, which are brown or gray sun-induced skin lesions, may appear on sun-exposed skin as a person gets older. It is common for consumers to wish to improve the appearance of such age-related skin imperfections such as wrinkles, crow's feet, age-spots, eye bags, and the like. Additionally, many consumers wish to improve the appearance of, or hide, other skin imperfections such as acne, scars, enlarged pores, and so on, which may not be related to aging.

While topical cosmetic formulations such as foundation or concealer types of make-up may improve the appearance of some skin imperfections, such formulations are not lasting and cannot reduce the appearance of more pronounced skin imperfections, such as deep wrinkles or scars. Further, while some cosmetic formulations may include an ingredient to reduce the appearance of imperfections over time, such as an anti-wrinkle cream, such formulations may take a long time for results to be noticeable, and may also be ineffective to reduce the appearance of more pronounced skin imperfections.

As an alternate to topical cosmetic formulations, more invasive techniques such as surgery, fillers, or laser resurfacing of the skin may provide longer-lasting effects and can treat prominent imperfections. However, many consumers either cannot afford, or do not wish, to undergo such drastic cosmetic treatments.

As such, there is a consumer desire for topical cosmetic formulations that are effective at reducing the appearance of skin imperfections.

### SUMMARY

The disclosure relates to compositions and methods for improving the appearance of the skin.

In one embodiment, the disclosure relates to compositions for tightening the skin, said compositions comprising (A) (a) at least one vinyl-terminated polysiloxane and at least one hydride-functionalized polysiloxane, and (b) at least one filler; and (B) (a) at least one vinyl-terminated polysiloxane; (b) at least one filler, and (c) at least one metal catalyst, wherein the viscosity of the at least one vinyl-terminated polysiloxane ranges from about 50,000 to about 100,000 cSt at 25° C., and wherein the specific surface area of the at least one filler is greater than about 300 m²/g.

In further embodiments, the disclosure relates to a skin tightening film formed from the compositions of the disclosure, having a Young Modulus greater than about 500 kPa.

In yet further embodiments, the disclosure relates to methods for improving the appearance of the skin, said methods comprising forming a film on the skin by applying a composition onto the skin, said composition comprising (A) (a) at least one vinyl-terminated polysiloxane and at least one hydride-functionalized polysiloxane, and (b) at least one filler; and (B) (a) at least one vinyl-terminated polysiloxane; (b) at least one filler, and (c) at least one metal catalyst, wherein the viscosity of the at least one vinyl-terminated polysiloxane ranges from about 50,000 to about 100,000 cSt at 25° C., and wherein the specific surface area of the at least one filler is greater than about 300 m²/g, wherein the film has a Young Modulus greater than about 500 kPa.

### DETAILED DESCRIPTION

In various embodiments, the disclosure relates to compositions and methods for improving the appearance of the skin. The compositions may be effective at reducing the appearance of skin imperfections. In various embodiments, the compositions may improve the appearance of the skin by forming a film on the skin that has a Young Modulus greater than that of skin, and thus has the capability of tightening the skin. Additionally, in some embodiments, the film may blur or hide skin imperfections. Accordingly, the disclosure further relates to methods of improving the appearance of the skin by forming a film on the skin with the compositions described herein.

As used herein, the term “long-lasting” means that the film lasts for at least about 6 hours, such as at least about 12 hours, at least about 24 hours, at least about 48 hours, or at least about 72 hours, after the film is formed on the skin.

As used herein, the term “lasting” it is meant to convey that the film is substantially intact in place on the skin.

As used herein, the term “forms quickly” means that the film forms within less than about 20 minutes, such as less than about 15 minutes, or less than about 10 minutes, after the composition is applied to the skin.

As used herein, the term “blur” with regard to skin imperfections means that the visual appearance of the imperfection is less noticeable.

As used herein, the term “tighten” means that the film contracts in a manner that skin has a tighter feel to the user, and that reduces the visual appearance of wrinkles in the skin.

As used herein, the term “soft focus” means that the visual appearance of the skin is more homogenous and matte, leading to the blurring or hiding of skin imperfections.

As used herein, “durable” means the film will not easily rub off, or will not be removed by sweat, water, makeup, lotions, or the like, such that the film will remain substantially intact until removed by the user.

As used herein, the phrase “relative to the weight of the composition” with respect to the weight of a component is intended to indicate the total weight of the composition after parts (A) and (B) are mixed.

### Compositions

According to various embodiments, the compositions comprise two parts, parts (A) and (B). Part (A) comprises (a) two reactive silicone components: a vinyl-terminated polysiloxane and at least one hydride-functionalized polysiloxane, and (b) at least one filler; and part (B) comprises (a) one reactive silicone component: a vinyl-terminated polysiloxane, (b) at least one filler, and (c) at least one metal catalyst.

### Reactive Components

The compositions comprise, as reactive components, vinyl-terminated polysiloxanes and hydride-functionalized polysiloxanes. The molar ratio of the vinyl-terminated polysiloxanes to hydride-functionalized polysiloxanes ranges from about 1:1 to about 1:20 in various embodiments, for example about 1:3. The total amount of reactive components according to various embodiments of the disclosure ranges from about 10% to about 65%, such as from about 10% to about 55%, about 15% to about 45%, or about 20% to about 30% by weight, relative to the total weight of the composition.

Vinyl-Terminated Polysiloxane

Both part (A) and part (B) of the compositions comprise vinyl-terminated polysiloxanes as a reactive silicone component. The vinyl-terminated polysiloxanes of parts (A) and (B) in any composition may be identical or different, according to various exemplary embodiments.

The vinyl-terminated polysiloxanes useful according to embodiments of the disclosure include vinyl-terminated polysiloxanes with a viscosity at 25° C. ranging from about 50,000 cSt to about 100,000 cSt.

In various embodiments, the vinyl-terminated polysiloxanes may be chosen from those of formula I:

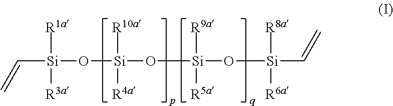

wherein:
each of R^{1a′}, R^{3a′}, R^{4a′}, R^{5a′}, R^{6a′}, R^{8a′}, R^{9a′}, and R^{10a′} are independently chosen from hydrogen, C₁-C₂₀ alkyl, C₂-C₂₀ alkenyl, C₅-C₁₀ aryl, hydroxyl, or C₁-C₂₀ alkoxyl; and
p and q are integers independently ranging from about 10 to about 6000.

The vinyl-terminated polysiloxanes may be chosen from those disclosed in U.S. Pat. No. 8,691,202. For example, without limitation, the vinyl-terminated polysiloxanes may be chosen from vinyl terminated polydimethylsiloxane; vinyl terminated diphenylsiloxane-dimethylsiloxane copolymers; vinyl terminated polyphenylmethylsiloxane, vinylphenylmethyl terminated vinylphenylsiloxane-phenylmethylsiloxane copolymer; vinyl terminated trifluoropropylmethylsiloxane-dimethylsiloxane copolymer; vinyl terminated diethylsiloxane-dimethylsiloxane copolymer: vinylmethylsiloxane-dimethylsiloxane copolymer, trimethylsiloxy terminated; vinylmethylsiloxane-dimethylsiloxane copolymers, silanol terminated; vinylmethylsiloxane-dimethylsiloxane copolymers, vinyl terminated; vinyl gums; vinylmethylsiloxane homopolymers; vinyl T-structure polymers; monovinyl terminated polydimethylsiloxanes; vinylmethylsiloxane terpolymers; vinylmethoxysilane homopolymers, and combinations thereof.

By way of example only, the vinyl-terminated polysiloxanes may be chosen from the vinyl-terminated polydimethylsiloxane (vinyl-dimethicone) sold under the name DMS-V46 by Gelest, with a viscosity at 25° C. of about 60,000 cSt.

The vinyl-terminated polysiloxane may be present in the composition in an amount ranging up to about 60%, such as from about 10% to about 50%, about 15% to about 40%, or about 20% to about 30% by weight, relative to the total weight of the composition.

The vinyl-terminated polysiloxane may be present in each of parts (A) and (B), independently, in an amount up to about 40%, such as from about 10% to about 30%, about 15% to about 25%, or about 17% to about 23% by weight, relative to the total weight of the part in which it is present.

Hydride-Functionalized Polysiloxane

Part A of the composition further comprises at least one hydride-functionalized polysiloxane. The viscosity of the hydride-functionalized polysiloxane can range up to about 100 cSt, such as about 10 to about 50 cSt, or about 25 cSt at 25° C.

In various embodiments, the hydride-functionalized polysiloxane may be chosen from those of formula II:

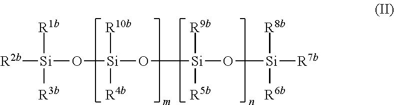

wherein:
R^{1b}, R^{2b}, R^{3b}, R^{4b}, R^{5b}, R^{6b}, R^{7b}, R^{8b}, R^{9b} and R^{10b} are each independently chosen from hydrogen, C₁₋₂₀ alkyl, C₂₋₂₀ alkenyl, C₅₋₁₀ aryl, hydroxyl or C₁₋₂₀ alkoxy,
and m and n are each independently an integer ranging from about 10 to about 6000,
with the proviso that at least one of R^{1b}, R^{2b}, R^{3b}, R^{4b}, R^{5b}, R^{6b}, R^{7b}, R^{8b}, R^{9b} and R^{10b} is hydrogen.

The hydride-functionalized polysiloxane may be chosen from those disclosed in U.S. Pat. No. 8,691,202. For example, without limitation, the hydride-functionalized polysiloxane may, in at least certain exemplary embodiments, be chosen from hydride terminated polydimethylsiloxane; polyphenyl-(dimethylhydrosiloxy)siloxane, hydride terminated; methylhydrosiloxane-phenylmethylsiloxane copolymer, hydride terminated; methylhydrosiloxane-dimethylsiloxane copolymers, trimethylsiloxy terminated; polymethylhydrosiloxanes, trimethylsiloxy terminated; polyethylhydrosiloxane, triethylsiloxane, methylhydrosiloxane-phenyloctylmethylsiloxane copolymer; methylhydrosiloxane-phenyloctylmethylsiloxane terpolymer, and combinations thereof.

By way of example only, the hydride-functionalized polysiloxanes may be chosen from polydimethyl methylhydrosiloxane sold under the name PS123 by United Chemical Technologies.

The hydride-functionalized polysiloxane may be present in the composition in an amount ranging from about 0.01% to about 3%, such as about about 0.05% to about 2%, about 0.1% to about 1%, or about 0.1% to about 0.5% by weight, relative to the weight of the composition.

### Filler

The compositions comprise at least one filler in each of parts (A) and (B). The fillers may be mineral or organic in nature, and of any shape. In various embodiments, the fillers may have a particle size greater than about 100 nm, and/or a specific surface area greater than about 300 m²/g.

By way of non-limiting example, fillers may be chosen from talc, mica, silica, silica surface-treated with a hydrophobic agent, fumed silica, kaolin, polyamide (Nylon®) powders (e.g. Orgasol® from Atochem), polyurethane powders, poly-β-alanine powder and polyethylene powder, powders of tetrafluoroethylene polymers (Teflon®), lauroyllysine, starch, boron nitride, hollow polymer microspheres such as those of polyvinylidene chloride/acrylonitrile, for instance Expancel® (Nobel Industrie) or of acrylic acid copolymers (Polytrap® from the company Dow Corning) and silicone resin microbeads (Tospearls® from Toshiba, for example), elastomeric polyorganosiloxane particles, precipitated calcium carbonate, magnesium carbonate, magnesium hydrogen carbonate, hydroxyapatite, hollow silica microspheres (Silica Beads® from Maprecos), glass or ceramic microcapsules, and metal soaps derived from organic carboxylic acids containing from 8 to 22 carbon atoms and preferably from 12 to 18 carbon atoms, for example zinc stearate, magnesium stearate or lithium stearate, zinc laurate or magnesium myristate.

In at least certain embodiments, the at least one filler may be chosen from hydrophobic silica aerogel particles. Silica aerogels are porous materials obtained by replacing (by drying) the liquid component of a silica gel with air. Hydrophobic silica aerogel particles useful according to embodiments of the disclosure include silylated silica (INCI name: silica silylate) aerogel particles. The preparation of hydrophobic silica aerogel particles that have been surface-modified by silylation is described more fully in U.S. Pat. No. 7,470,725, incorporated by reference herein.

In various embodiments, aerogel particles of hydrophobic silica surface-modified with trimethylsilyl groups may be chosen. For example, the aerogel sold under the name VM-2260® by the company Dow Corning, the particles of which have an average size of about 1000 microns and a specific surface area per unit of mass ranging from 600 to 800 m²/g, or the aerogel sold under the name VM-2270®, also by the company Dow Corning, the particles of which have an average size ranging from 5 to 15 microns and a specific surface area per unit of mass ranging from 600 to 800 m²/g, may be chosen. In other embodiments, the aerogels sold by the company Cabot under the names Aerogel TLD 201®, Aerogel OGD 201®, and Aerogel TLD 203®, CAB-O-SIL TS-530, CAB-O-SIL TS-610, CAB-O-SIL TS-720, Enova Aerogel MT 1100®, and Enova Aerogel MT 1200®, may be chosen.

Optionally, mixtures of fillers may be present in the compositions according to the disclosure. For example, a mixture of different aerogel particles, or of an aerogel and a different type of filler, may be used.

The at least one filler may be present in the composition in a total amount ranging from up to about 8% by weight, such as, for example from about 1% to about 7%, from about 2% to about 6%, or from about 3% to about 5%, by weight, relative to the total weight of the composition.

The at least one filler may be present in each of parts (A) and (B), independently, in an amount up to about 6% by weight, such as, for example from about 0.5% to about 5%, from about 1% to about 4%, or from about 1.5% to about 3%, by weight, relative to the total weight of the part in which it is present.

In at least certain embodiments, the amount of the at least one filler and the at least one vinyl-terminated polysiloxane in parts (A) and (B) may be the same or substantially the same. This may allow the viscosity of parts (A) and (B) to be the same or substantially the same, thereby aiding in formation of a homogenous composition.

### Catalyst

Part (B) of the composition also comprises at least one metal catalyst. The catalyst may be chosen from any metal catalyst, such as, for example, platinum catalysts, rhodium catalysts, and tin catalysts.

By way of non-limiting example, platinum catalysts may be chosen from platinum carbonyl cyclovinylmethylsiloxane complexes, platinum divinyltetramethyldisiloxane complexes, platinum cyclovinylmethylsiloxane complexes, platinum octanaldehyde/octanol complexes and combinations thereof. A non-limiting example of a rhodium catalyst includes Tris (dibutylsulfide) Rhodium utichloride. Non-limiting examples of tin catalysts include tin II octoate, Tin II neodecanoate, dibutyltin diisooctylmaleate, di-n-butylbis(2,4 pentanedionate)tin, di-n-butylbutoxychlorotin, dibutyltin dilaurate, dimethyltin dineodecanoate, dimethylhydroxy(oleate)tin and tin II oleate. The metal catalyst may, for example, be PC075.3 from United Cheical Technolgies.

The metal catalyst may be present in the composition in an amount up to about 5%, such as up to about 4%, up to about 3%, up to about 2%, up to about 1.5%, up to about 1%, or up to about 0.5% by weight, relative to the weight of the composition.

### Additional Components

The compositions further comprise additional components such as volatile solvents, emulsifiers, colorants, silicone elastomers, and humectants. Any additional components may independently be present in part (A), part (B), or both.

Volatile Solvents

The volatile solvents may be chosen, in various embodiments, from volatile solvents having a vapor pressure greater than about 50 Pa at 25° C. and atmospheric pressure, such as greater than about 100 Pa. By way of example, the volatile solvent may be chosen from water, a non-silicone organic solvent or a silicone organic solvent, or mixtures thereof.

Volatile non-silicone organic solvents may be chosen from, for example, volatile C1-C4 alkanols such as ethanol or isopropanol; volatile C5-C7 alkanes such as n-pentane, hexane, cyclopentane, 2,3-dimethylbutane, 2,2-dimethylbutane, 2-methylpentane or 3-methylpentane; esters of liquid C1-C20 acids and of volatile C1-C8 alcohols such as methyl acetate, n-butyl acetate, ethyl acetate, propyl acetate, isopentyl acetate or ethyl 3-ethoxypropionate; ketones that are liquid at room temperature and volatile, such as methyl ethyl ketone, methyl isobutyl ketone, diisobutyl ketone, isophorone, cyclohexanone or acetone; volatile polyols such as propylene glycol; volatile ethers such as dimethoxymethane, diethoxyethane or diethyl ether; volatile glycol ethers such as 2-butoxyethanol, butyl diglycol, diethylene glycol monomethyl ether, propylene glycol n-butyl ether or propylene glycol monomethyl ether acetate; volatile hydrocarbon-based oils such as volatile hydrocarbon-based oils containing from 8 to 16 carbon atoms, and mixtures thereof, and especially branched C8-C16 alkanes, for instance C8-C16 isoalkanes (also known as isoparaffins), isododecane, isodecane and, for example, the oils sold under the trade names Isopar or Permethyl, and mixtures thereof. Mention may also be made of isohexyl or isodecyl neopentanoate; volatile C4-C10 perfluoroalkanes such as dodecafluoropentane, tetradecafluorohexane or decafluoropentane; volatile perfluorocycloalkyls such as perfluoromethylcyclopentane, 1,3-perfluorodimethylcyclohexane and perfluorodecalin, sold, respectively, under the names Flutec PC1, Flutec PC3 and Flutec PC6 by the company F2 Chemicals, and also perfluorodimethylcyclobutane and perfluoromorpholine.

Volatile silicone solvents may be chosen from, for example, low-viscosity silicone compounds chosen from linear or cyclic silicones containing from 2 to 7 silicon atoms, these silicones optionally comprising alkyl or alkoxy groups containing from 1 to 10 carbon atoms, for example trisiloxanes, tetrasiloxanes, pentasiloxanes, hexasiloxanes, heptasiloxanes, octasiloxanes, and decasiloxanes, including but not limited to octamethylcyclotetrasiloxane, decamethylcyclopentasiloxane, dodecamethylcyclohexasiloxane, heptamethylhexyltrisiloxane, heptamethylethyltrisiloxane, heptamethyloctyltrisiloxane, octamethyltrisiloxane and decamethyltetrasiloxane, and mixtures thereof. According to various exemplary embodiments, the volatile solvent compound is chosen from trisiloxanes, cyclopentadimethylsiloxane, and/or dodecamethylcyclohexasiloxane.

Non-limiting examples include the decamethylcyclopentasiloxane sold under the name DC-245, the octamethyltrisiloxane sold under the name DC-200 Fluid 1 cSt, and the decamethyltetrasiloxane sold under the name DC-200 Fluid 1.5 cSt, all by the company Dow Corning.

In various embodiments, the at least one volatile solvent may be present in the composition in an amount up to about 90%, such as up to about 80%, up to about 70%, up to about 60%, up to about 50%, up to about 40%, or up to about 30% by weight, relative to the total weight of the composition.

Emulsifiers

Non-limiting examples of emulsifiers include amphoteric, anionic, cationic and nonionic emulsifiers, used alone or as a mixture. For example, emulsifiers may be chosen from sorbitan, glycerol or sugar alkyl esters or ethers; silicone surfactants, for instance dimethicone copolyols, such as the mixture of cyclomethicone and of dimethicone copolyol (sold under the name DC 5225 C by the company Dow Corning), and alkyldimethicone copolyols such as laurylmethicone copolyol (sold under the name Dow Corning 5200 Formulation Aid by the company Dow Corning); cetyldimethicone copolyol (e.g. the product sold under the name Abil EM 90R by the company Evonik), and the mixture of cetyldimethicone copolyol, of polyglyceryl isostearate (4 mol) and of hexyl laurate (e.g. product sold under the name Abil WE O9 by the company Evonik). One or more co-emulsifiers may also be added thereto, which may be chosen for example from the group comprising polyol alkyl esters.

Exemplary polyoxyalkylenated silicone elastomers useful as emulsifiers include those disclosed in U.S. Pat. Nos. 5,236,986, 5,412,004, 5,837,793 and 5,811,487. For example, emulsifiers may be chosen from those available from Shin Etsu: KSG-16 dimethicone (and) dimethicone/vinyl dimethicone corpsspolymer, KSG-21 (at 27% in active material) (INCI name: Dimethicone/PEG-10 Dimethicone vinyl dimethicone crosspolymer), KSG-20 (at 95% in active material) (INCI name: PEG-10 Dimethicone Crosspolymer), KSG-30, (at 100% in active material) (INCI name: Lauryl PEG-15 Dimethicone vinyl dimethicone crosspolymer), KSG-31 (at 25% in active material) (INCI name: Lauryl PEG-15 Dimethicone vinyl dimethicone crosspolymer), KSG-32, KSG-42, KSG-320 or KSG-30 (at 25% in active material) (INCI name: Lauryl PEG-15 Dimethicone vinyl dimethicone crosspolymer), KSG-33: Lauryl PEG-15 (at 20% in active material) (Dimethicone vinyl dimethicone crosspolymer), KSG-210 (at 25% % in active material) (INCI name: Dimethicone/PEG-10/15 crosspolymer), KSG-310 (lauryl modified polydimethylsiloxane polyoxyethylenated in mineral oil), KSG-330 and KSG-340 (PEG-15/lauryl dimethicone crosspolymer, X-226146 (at 32% % in active material) (INCI name: Dimethicone/PEG-10 Dimethicone vinyl dimethicone crosspolymer); and those available from Dow Corning: DC9010 (at 9% in active material) and DC9011 (at 11% in active material) (INCI name: PEG-12 dimethicone crosspolymer), DC9040 cyclopentasiloxane (and) dimethicone crosspolymer, DC9041 dimethicone (and) dimethicone crosspolymer; and mixtures thereof.

In other embodiments, polyglycerolated silicone elastomers may be chosen as emulsifiers. Examples of such compounds are provided in WO 2004/024798. For example, emulsifiers may be chosen from those available from Shin Etsu: KSG-710 (at 25% in active material, INCI name: dimethicone/polyglycerin-3 crosspolymer); and KSG-820, KSG-830 and KSG-840, all of which are dimethicone/polvaleverin-3 crosspolymer (INCI), but in different diluents, 820 is in isododecane, 830 is in triethyl hexanoin and 840 is in squalene.

Colorants

The composition may further include at least one colorant, for example to create a colored film on the skin, which may be useful to hide certain skin imperfections. In various embodiments, the at least one colorant may be chosen from dyes, pigments, and nacres.

The at least one colorant may, for example, be chosen from dyes. Non-limiting examples of dyes include Sudan Red, D & C Red 17, D & C Green 6, β-carotene, soybean oil, Sudan Brown, D & C Yellow 11, D & C Violet 2, D & C Orange 5, quinoline yellow and annatto.

In various embodiments, the at least one colorant may be chosen from pigments. As used herein, the term “pigments” is intended to mean white or colored, mineral or organic particles which are insoluble in the composition in which they are present, and which are intended to color and/or opacify the resulting film.

By way of example, inorganic pigments that may be used include titanium oxides, zirconium oxides, cerium oxides, zinc oxides, iron oxides, chromium oxides, ferric blue, manganese violet, ultramarine blue, and chromium hydrate. For example, pigments may be chosen from titanium dioxide and red, black, and/or yellow iron oxide, as well as mixtures thereof.

In further embodiments, pigments with a structure that may be, for example, of silica microspheres containing iron oxide type, may be used. An example of a pigment having this structure is the product sold by the company Miyoshi under the reference PC Ball PC-LL-100 P, constituted of silica microspheres containing yellow iron oxide.

By way of further example, organic pigments that may be used include nitroso, nitro, azo, xanthene, pyrene, quinoline, anthraquinone, triphenylmethane, fluorane, phthalocyanin, metal complex, isoindolinone, isoindoline, quinacridone, perinone, perylene, diketopyrrolopyrrole, indigo, thioindigo, dioxazine, triphenylmethane and quinophthalone compounds. For example, the organic pigments may be chosen from carmine lake, carbon black, aniline black, azo yellow, quinacridone, phthalocyanine blue, the blue pigments codified in the Color Index under the references CI 42090, 69800, 69825, 73000, 74100 and 74160, the yellow pigments codified in the Color Index under the references CI 11680, 11710, 15985, 19140, 20040, 21100, 21108, 47000 and 47005, the green pigments codified in the Color Index under the references CI 61565, 61570 and 74260, the orange pigments codified in the Color Index under the references CI 11725, 15510, 45370 and 71105, the red pigments codified in the Color Index under the references CI 12085, 12120, 12370, 12420, 12490, 14700, 15525, 15580, 15620, 15630, 15800, 15850, 15865, 15880, 17200, 26100, 45380, 45410, 58000, 73360, 73915 and 75470, and the pigments obtained by oxidative polymerization of indole or phenolic derivatives as described in patent FR 2 679 771.

Nacres may be chosen from white pearlescent pigments such as mica coated with titanium or with bismuth oxychloride, colored pearlescent pigments such as titanium mica with iron oxides, titanium mica with in particular ferric blue or the chromium oxide, titanium mica with an organic pigment of the abovementioned type, and pearlescent pigments based on bismuth oxychloride.

The one or more colorants may optionally be included in the composition in an amount up to about 5%, such as up to about 4.5%, up to about 4%, up to about 3.5%, up to about 3%, up to about 2.5%, up to about 2%, up to about 1.5%, up to about 1%, up to about 0.75%, up to about 0.5%, up to about 0.25%, up to about 0.2%, or up to about 0.1%, weight, relative to the weight of the composition.

Silicone Elastomer

The composition may further optionally comprise at least one silicone elastomer. It may, in at least certain embodiments, be advantageous to choose a silicone elastomer having greater than 1% active material (AM), such as greater than 2% AM.

The at least one silicone elastomer may, for example, be chosen from at least one silicone crosspolymer dispersed in at least one oil. The at least one silicone crosspolymer may, in certain embodiments, be chosen from dimethicone crosspolymers, such as dimethicone/vinyl dimethicone crosspolymers and dimethicone/phenyl vinyl dimethicone crosspolymers. In other embodiments, the silicone cross-polymer may be modified by one or more groups chosen from alkyl, polyether, polyglycerin groups. For instance, the alkyl modified silicone cross-polymers may be chosen from vinyl dimethicone/lauryl dimethicone cross-polymers, cetearyl dimethicone cross-polymers, and C₃₀-C₄₅ alkyl cetearyl dimethicone cross-polymers. Non-limiting examples of polyether modified silicone cross-polymers include dimethicone/PEG-10/15 cross-polymers. Exemplary alkyl and polyether modified silicone cross-polymers may be chosen, for example, from PEG-10/lauryl dimethicone cross-polymers and PEG-15/lauryl dimethicone cross-polymers. Exemplary polyglycerin modified silicone cross-polymers include dimethicone/polyglycerin-3 cross-polymers and lauryl dimethicone/polyglycerin-3 cross-polymers.

In at least certain embodiments, the silicone polymers do not comprise polyethylene glycol or polypropylene groups, or hydrophilic moieties. Optionally, the silicone elastomer may be chosen from the silicone organic blends isododecane (and) dimethicone crosspolymer (18% AM) sold under the name EL-8040 ID or dimethicone/bis-isobutyl PPG-20 crosspolymer (17% AM in isododecane) sold under the name EL-8050 ID, by Dow Corning; or isododecane (and) vinyldimethyl/trimethylsiloxysilicate stearyl dimethicone crosspolymer (20% AM in isododecane), sold under the name GEL BELSIL RG90 by Wacker.

The silicone crosspolymer may be dispersed in at least one oil. In certain embodiments, the oil may be chosen from silicone oils, such as cyclic and linear organopolysiloxanes. Cyclic organopolysiloxanes may include, for example, cyclotetrasiloxane; cyclopentasiloxane; and methylated cyclic organopolysiloxanes, for example, octamethylcyclotetrasiloxane and decamethylcyclopentasiloxane. Non-limiting examples of linear organopolysiloxanes include low molecular weight dimethicones; high molecular weight dimethicones; alkyl derivatives of linear organopolysiloxanes, for example, cetyl dimethicone and lauryl trimethicone; aryl derivatives of linear organopolysiloxanes, for example, phenyl trimethicone; and hydroxylated derivatives of linear organopolysiloxanes, for example, dimethiconol. In other embodiments, the oil may be chosen from organic oils, such as mineral oil; linear and branched alkanes, for example, isododecane; triethylhexanoin; and squalane.

The at least one silicone crosspolymer may, in some embodiments, comprise from about 5% to about 35% by weight, relative to the total weight of the silicone elastomer blend, for example, from about 10% to about 20% by weight, or from about 25% to about 35% by weight, or from about 20% to about 30% by weight. The at least one oil may comprise from about 65% to about 95% by weight, relative to the total weight of the silicone elastomer blend, such as from about 80% to about 90% by weight, or from about 65% to about 75% by weight, or from about 70% to about 80% by weight.

In various exemplary embodiments, the silicone elastomer blend comprises from about 20% to about 30% of dimethicone/vinyl dimethicone cross-polymer. In further exemplary embodiments, the silicone elastomer blend comprises from about 70% to about 80% by weight of dimethicone. In yet further exemplary embodiments, the silicone elastomer blend comprises from about 20% to about 30% of dimethicone/vinyl dimethicone cross-polymer and from about 70% to about 80% by weight dimethicone.

For example, silicone elastomers sold under the name KSG-16 dimethicone (and) dimethicone/vinyl dimethicone corpsspolymer, KSG-21 (at 27% in active material) INCI name: Dimethicone/PEG-10 Dimethicone vinyl dimethicone crosspolymer), KSG-20 (at 95% % in active material) INCI name: PEG-10 Dimethicone Crosspolymer), KSG-30, (at 100% % in active material) INCI name: Lauryl PEG-15 Dimethicone vinyl dimethicone crosspolymer), KSG-31 (at 25% in active material) INCI name: Lauryl PEG-15 Dimethicone vinyl dimethicone crosspolymer), KSG-32 or KSG-42 or KSG-320 or KSG-30 (at 25% in active material) INCI name: Lauryl PEG-15 Dimethicone vinyl dimethicone crosspolymer), KSG-33: Lauryl PEG-15 (at 20% in active material) Dimethicone vinyl dimethicone crosspolymer), KSG-210 (at 25% in active material) INCI name: Dimethicone/PEG-10/15 crosspolymer), KSG-310: lauryl modified polydimethylsiloxane polyoxyethylenated in mineral oil, KSG-330 and KSG-340: PEG-15/lauryl dimethicone crosspolymer, and X-226146 (at 32% % in active material) INCI name: Dimethicone/PEG-10 Dimethicone vinyl dimethicone crosspolymer), all by Shin Etsu; DC9010 (at 9% in active material) and DC9011 (at 11% in active material) INCI name: PEG-12 dimethicone crosspolymer), DC9040 cyclopentasiloxane (and) dimethicone crosspolymer, and DC9041 dimethicone (and) dimethicone crosspolymer, all by Dow Corning; or the products sold under the VELVESIL product line by Momentive, such as VELVESIL 125 and VELVESIL DM, may be chosen.

Other examples of silicone elastomers include KSG-710 (at 25% in active material, INCI name: dimethicone/polyglycerin-3 crosspolymer); and KSG-820, KSG-830 and KSG-840, all of which are dimethicone/polvaleverin-3 crosspolymer (INCI), but in different diluents, 820 is in isododecane, 830 is in triethyl hexanoin, and 840 is in squalene, all by Shin Estu.

The at least one silicone elastomer may optionally be included in the composition in an amount up to about 10%, such as up to about 8%, up to about 5%, about 4.5%, up to about 4%, up to about 3.5%, up to about 3%, up to about 2.5%, up to about 2%, up to about 1.5%, up to about 1%, up to about 0.75%, up to about 0.5%, up to about 0.25%, up to about 0.2%, or up to about 0.1%, by weight, relative to the weight of the composition. In certain embodiments, the at least one silicone elastomer may be present in an amount ranging from about 1% to about 10%, such as about 2% to about 8%, about 3% to about 6%, or about 4% to about 5%, by weight, relative to the total weight of the composition.

Humectants

Optionally, compositions according to the disclosure may comprise at least one humectants (emollient) or moisturizing agent.

By way of example only, humectants or moisturizing agents may be chosen from polyhydroxy compounds including but not limited to glycerin and glycols such as, for example, propylene glycol, butylene glycol, dipropylene glycol and diethylene glycol, glycol ethers such as monopropylene, dipropylene and tripropylene glycol alkyl(C₁-C₄)ethers, monoethylene, diethylene and triethylene glycol.

The at least one humectant may be present in the composition in an amount up to about 20%, such as up to about 15%, up to about 14%, up to about 13%, up to about 12%, up to about 11%, up to about 10%, up to about 9%, up to about 8%, up to about 7%, up to about 6%, up to about 5%, up to about 4%, up to about 3%, up to about 2%, up to about 1%, or up to about 0.5%, by total weight of the composition.

### Film

When the compositions according to the disclosure are applied to the skin, the components form a matrix that creates a film on the skin. The films formed by the compositions described herein form quickly, are long-lasting and durable, and have optical properties that are advantageous for a skin-tightening film, such as transparency, matte effect, and a soft focus effect which helps to blur skin imperfections so that they are less noticeable.

Additionally, as discussed above, the compositions according to the disclosure form a film that is stiffer than, and thus capable of tightening, human skin. Human skin has a Young Modulus in the range of 10 kPa to 100 kPa; thus, a film for tightening the skin should have a Young Modulus of greater than 100 kPa. The films that are formed by the compositions have Young Modulus' greater than about 250 kPa (0.25 MPa) in some embodiments, such as greater than about 500 kPa (0.5 MPa) in some embodiments, greater than 1000 kPa (1 MPa) in some embodiments, greater than 5000 kPa (5 MPa) in some embodiments, and even greater than 10,000 kPa (10 MPa) in some embodiments. By way of example only, the film may have a Young Modulus ranging from about 500 kPa to about 50,000 kPa, such as about 500 kPa to about 25,000 kPa, about 500 kPa to about 10,000 kPa, about 500 kPa to about 5000 kPa, about 500 kPa to about 2000 kPa, or about 500 kPa to about 1000 kPa.

As such, the amounts and components of the composition should be chosen to provide a film on the skin that is capable of tightening the skin, while also blurring skin imperfections.

The films may be formed quickly, for example within less than about 30 minutes, less than about 20 minutes, less than about 10 minutes, or less than about 5 minutes, after the composition is applied to the skin.

Films according to the disclosure may be long-lasting. For example, once the composition is applied to the skin and a film is formed, the film may remain substantially intact on the skin for a period of at least about 12 hours, such as at least about 24 hours, at least about 48 hours, or at least about 72 hours.

The films may also be durable. For example, the film may not rub off, may not come off with sweat, or when the film is contacted by water, makeup, lotions, or other products that the user may wish to put on the skin.

### Methods

Methods of improving the appearance of the skin are also disclosed, said methods comprising applying a composition according to the disclosure onto the skin in order to form a film on the skin. Methods comprise tightening the skin, e.g. to get rid of wrinkles, eye bags, etc., and/or blurring or hiding skin imperfections, e.g. to camouflage pimples, pores, dark spots, etc.

The parts (A) and (B) of the composition may be applied separately to the skin, as a 2-step process, or they may be applied simultaneously, for example when dispensed from a double chamber container and mixed as the composition is applied to the skin. In such embodiments, it may be useful to have the amounts of vinyl-terminated polysiloxanes and fillers in parts (A) and (B) be the same or substantially the same. In various embodiments, it may be advantageous to apply each part in approximately the same amount, so that the parts (A):(B) are in approximately a 1:1 ratio.

### Kits

In further embodiments, the disclosure relates to kits containing the compositions according to the disclosure, where parts (A) and (B) are packaged in separate compartments, such as, for example, a dual chamber container optionally with a dispenser.

It to be understood that, as used herein the terms “the,” “a,” or “an,” mean “at least one,” and should not be limited to “only one” unless explicitly indicated to the contrary. Thus, for example, reference to “a portion” includes examples having two or more such portions unless the context clearly indicates otherwise.

It should be understood that all patents and published patent applications referenced are incorporated herein in their entireties.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that any particular order be inferred.

While various features, elements or steps of particular embodiments may be disclosed using the transitional phrase “comprising,” it is to be understood that alternative embodiments, including those that may be described using the transitional phrases “consisting” or “consisting essentially of,” are implied. Thus, for example, implied alternative embodiments to a method that comprises A+B+C include embodiments where a method consists of A+B+C and embodiments where a method consists essentially of A+B+C. As described, the phrase “at least one of A, B, and C” is intended to include “at least one A or at least one B or at least one C,” and is also intended to include “at least one A and at least one B and at least one C.”

All ranges and amounts given herein are intended to include subranges and amounts using any disclosed point as an end point. Thus, a range of “1% to 10%, such as 2% to 8%, such as 3% to 5%,” is intended to encompass ranges of “1% to 8%,” “1% to 5%,” “2% to 10%,” and so on. All numbers, amounts, ranges, etc., are intended to be modified by the term “about,” whether or not so expressly stated. Similarly, a range given of “about 1% to 10%” is intended to have the term “about” modifying both the 1% and the 10% endpoints.

It is understood that when an amount of a component is given, it is intended to signify the amount of the active material.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present disclosure.

Notwithstanding that the numerical ranges and parameters setting forth the broad scope of the disclosure are approximations, unless otherwise indicated the numerical values set forth in the specific examples are reported as precisely as possible. Any numerical value, however, inherently contains certain errors necessarily resulting from the standard deviation found in their respective testing measurements. The example that follows serves to illustrate embodiments of the present disclosure without, however, being limiting in nature.

The compositions and methods according to the present disclosure can comprise, consist of, or consist essentially of the elements and limitations described herein, as well as any additional or optional ingredients, components, or limitations described herein or otherwise known in the art.

It will be apparent to those skilled in the art that various modifications and variations can be made in the delivery system, composition and methods of the invention without departing from the spirit or scope of the invention. Thus, it is intended that the present invention cover the modifications and variations of this invention provided that they come within the scope of the appended claims and their equivalents.

### EXAMPLES

The following Examples are provided for illustrative purposes only, and are not intended to be limiting.

In each of the following examples, the amounts of components given are in terms of active material (AM).

### Dynamic Mechanical Analysis (DMA)

The determination of Young Modulus of the films for all Examples was as follows. Equal amount of Part A and B of each formula was dispensed, mixed, and drawn on a Teflon plate using a 8 mil draw down bar. After a minimum of 3 hours, a 5.3 mm wide film band was cut, detached and mounted on the clamps of a TA Instruments Dynamic Mechanical Analyzer (DMA Q800). A stress/strain procedure was run at room temperature, with a pre-load force of 0.001N and at a rate of 5% per minute. (min N=3).

### Shear Storage Modulus

Equal amount of Part A and B of each formula was dispensed onto the plate of TA Instruments Rheometer. The 2 parts were mixed and the geometry (20 mm standard steel parallel plate) lowered down to 500 um (gap). A pre-shear conditioning step was performed for 10 sec at a shear rate of 10 1/s at 32° C., followed by a mixed procedure comprising a 15 min peak hold at 0.01 1/s shear rate and 32° C., followed by a strain sweep from 0.01% to 1000% strain at angular frequency of 1 rad/s. The reported shear storage modulus (kPa) was measured at 18 minutes (1% strain).

### Haze and Transparency-BYK Haze-Guard

Equal amount of Part A and B of each formula was dispensed, mixed, and drawn on a polyester film (BYK) using a 6 mil draw down bar. Haze and transparency were evaluated at 3 hours using a BYK Haze-Guard. (N=3).

### Gloss—BYK Glossmeter

Equal amount of Part A and B of each formula was dispensed, mixed and drawn on a contrast card using a 6 mil draw down bar. Gloss was measured at 10 minutes with a BYK Glossmeter. A measured value below 10 at 60 degree is low gloss or matte, and above 10 at 60 degrees is glossy or non-matte. (N=3).

### Stickiness

Equal amount of Part A and B of each formula was dispensed, mixed and drawn on a contrast card using a 6 mil draw down bar. After 3 hours, stickiness was evaluated with finger on a scale 1 to 3 (1—non sticky, 2—sticky, 3—very sticky).

### Viscosity

The formula was dispensed onto the plate of the TA instrument Rheometer, and the geometry (20 mm standard steel parallel plate) was lowered down to 1000 um (gap). A flow procedure consisting of ramping the shear rate from 0.01 1/s to 1000 1/s was performed at 25° C. The reported viscosity (Pa·s) was measured at a shear rate of 118 1/s.

### Example 1: Selection of Filler

The compositions were prepared as follows. Components 1-7 were mixed for 3 min at 2500 rpm. After pre-melting component 10, components 8-11 were added to the mix, and mixed for 2 minutes at 2500 rpm (Mixture A). In a separate container, components 12-15 were stirred for 10 minutes at 72° C. (Mixture B). Mixture B was added to mixture A under strong agitation (700 rpm). The mixing speed was increased progressively while adding mixture B, to reach 1000 rpm at the end. The formula was finalized for 10 minutes at 1100 rpm and confirmed as homogeneous.

The following Table 1 shows the comparison of the film formed from a composition prepared according to the disclosure (Ex. 1-1), and three comparative compositions (Ex. 1C-1, 1C-2, 1C-3).

TABLE 1
Demonstration of superior performance for filler

| | | Ex. 1-1 | | Ex. 1C-1 | | Ex. 1C-2 | | Ex. 1C-3 | |
|---|---|---|---|---|---|---|---|---|---|
| INCI name | Component # | 1-1A | 1-1B | 1C-1A | 1C-1B | 1C-2A | 1C-2B | 1C-3A | 1C-3B |
| VINYL-TERMINATED | 1 | 20 | 20 | 20 | 20 | 20 | 20 | 20 | 20 |
| POLYDIMETHYLSILOXANE | | | | | | | | | |
| POLYDIMETHYL- | 2 | 0.4 | | 0.4 | | 0.4 | | 0.4 | |
| METHYLHYDROSILOXANE | | | | | | | | | |
| PLATINUM- | 3 | | 0.7 | | 0.7 | | 0.7 | | 0.7 |
| DIVINYLTETRAMETHYLDISILOXANE | | | | | | | | | |
| COMPLEX | | | | | | | | | |
| TRISILOXANE | 4 | 38.6 | 38.3 | 38.6 | 38.3 | 38.6 | 38.3 | 31.1 | 30.8 |
| SILICA SILYLATE (1) | 5 | 2.5 | 2.5 | | | | | | |
| SILICA SILYLATE (2) | 6 | 1.67 | 1.67 | 4.17 | 4.17 | 1.67 | 1.67 | 1.67 | 1.67 |
| SILICA SILYLATE (3) | 7 | | | | | 2.5 | 2.5 | 10 | 10 |
| DIMETHICONE (and) | 8 | 2.17 | 2.17 | 2.17 | 2.17 | 2.17 | 2.17 | 2.17 | 2.17 |
| DIMETHICONE/PEG-10/15 | | | | | | | | | |
| CROSSPOLYMER | | | | | | | | | |
| DIMETHICONE (and) PEG/PPG- | 9 | 0.57 | 0.57 | 0.57 | 0.57 | 0.57 | 0.57 | 0.57 | 0.57 |
| 18/18 DIMETHICONE | | | | | | | | | |
| PEG-30 DIPOLYHYDROXYSTEREATE | 10 | 0.54 | 0.54 | 0.54 | 0.54 | 0.54 | 0.54 | 0.54 | 0.54 |
| PEG-10 DIMETHICONE | 11 | 0.72 | 0.72 | 0.72 | 0.72 | 0.72 | 0.72 | 0.72 | 0.72 |
| WATER | 12 | 31.03 | 31.03 | 31.03 | 31.03 | 31.03 | 31.03 | 31.03 | 31.03 |
| MAGNESIUM SULFATE | 13 | 1 | 1 | 1 | 1 | 1 | 1 | 1 | 1 |
| CAPRYLYL GLYCOL | 14 | 0.3 | 0.3 | 0.3 | 0.3 | 0.3 | 0.3 | 0.3 | 0.3 |
| PHENOXYETHANOL | 15 | 0.5 | 0.5 | 0.5 | 0.5 | 0.5 | 0.5 | 0.5 | 0.5 |
| TOTAL | | 100 | 100 | 100 | 100 | 100 | 100 | 100 | 100 |

| | | | | |
|---|---|---|---|---|
| ASPECT | White | White cream | White fluid | White thick |
| | creamy fluid | | | cream |
| EASY TO APPLY | Yes | Yes | Ok | No |
| SHEAR STORAGE MODULUS | 5.6 | 3.8 | 2 | 12.8 |
| (kPa) at 18 min | | | | |
| YOUNG'S MODULUS (kPa) | 513.7 | 237.5 | 162 | 1000 |
| MATITY (gloss at 60 degree at 10 min) | Yes (2.6) | No (15.3) | No (25.4) | No (12.2) |
| HAZE | 98.4 | 50 | 48.1 | 38.53 |
| TRANSPARENCY | 84.73 | 86.96 | 87.11 | 86.53 |
| STICKINESS | 1 | 2 | 3 | 2 |

(1) DOW CORNING VM-2270 AEROGEL, 98% AM; Specific surface = 600-800 m²/g
(2) CABOT CAB-O-SIL TS-530, 100% AM; Specific surface = 205-245 m²/g
(3) EVONIK AEROSIL R8200, 100% AM; Specific surface = 135-185 m²/g

Table 1 demonstrates that for silica with specific surface below 300 m²/g, at same concentration (Ex. 1C-1 and 1C-2), the performance (shear storage modulus, young's modulus, matity, haze and stickiness) is not as good as Ex. 1-1. Increasing such a silica to a higher concentration (Ex. 1C-3) increases the shear storage modulus and young's modulus of the film, but does not improves the matity or the haze, and remains sticky.

### Example 2: Ratio of Vinyl-Terminated to Hydride-Functionalized Polysiloxanes

The compositions were prepared as follows. Components 1-6 were mixed for 3 minutes at 2500 rpm. After pre-melting component 9, components 7-10 were added to the mix, and mixed for 2 minutes at 2500 rpm (Mixture A). In a separate container, components 11-14 were stirred for 10 minutes at 72° C. (Mixture B). Mixture B was added to mixture A under strong agitation (700 rpm). The mixing speed was increased progressively while adding mixture B, to reach 1000 rpm at the end. The formula was finalized for 10 minutes at 1100 rpm and confirmed as homogeneous.

TABLE 2
Ratio of vinyl-terminated:hydride-functionalized polysiloxanes

| | | Ratio | | | | | |
|---|---|---|---|---|---|---|---|
| | | 1:3 | | 1:25 | | 1:70 | |
| | | Ex. 2-1 | | Ex. 2C-1 | | Ex. 2C-2 | |
| INCI name | Component # | 2-1A | 2-1B | 2C-1A | 2C-1B | 2C-2A | 2C-2B |
| VINYL-TERMINATED PDMS | 1 | 20.00 | 20.00 | 20.00 | 20.00 | 20.00 | 20.00 |
| POLYDIMETHYL- | 2 | 0.40 | | 3.33 | | 9.33 | |
| METHYLHYDROSILOXANE | | | | | | | |
| PLATINUM- | 3 | | 0.70 | | 0.70 | | 0.70 |
| DIVINYLTETRAMETHYLDISILOXANE | | | | | | | |
| COMPLEX | | | | | | | |
| TRISILOXANE | 4 | 38.60 | 38.30 | 35.67 | 38.30 | 29.67 | 38.30 |
| SILICA SILYLATE (1) | 5 | 2.50 | 2.50 | 2.50 | 2.50 | 2.50 | 2.50 |
| SILICA SILYLATE (2) | 6 | 1.67 | 1.67 | 1.67 | 1.67 | 1.67 | 1.67 |
| DIMETHICONE (and) | 7 | 2.17 | 2.17 | 2.17 | 2.17 | 2.17 | 2.17 |
| DIMETHICONE/PEG-10/15 | | | | | | | |
| CROSSPOLYMER | | | | | | | |
| DIMETHICONE (and) PEG/PPG-18/18 | 8 | 0.57 | 0.57 | 0.57 | 0.57 | 0.57 | 0.57 |
| DIMETHICONE | | | | | | | |
| PEG-30 DIPOLYHYDROXYSTEREATE | 9 | 0.54 | 0.54 | 0.54 | 0.54 | 0.54 | 0.54 |
| PEG-10 DIMETHICONE | 10 | 0.72 | 0.72 | 0.72 | 0.72 | 0.72 | 0.72 |
| WATER | 11 | 31.03 | 31.03 | 31.03 | 31.03 | 31.03 | 31.03 |
| MAGNESIUM SULFATE | 12 | 1.00 | 1.00 | 1.00 | 1.00 | 1.00 | 1.00 |
| CAPRYLYL GLYCOL | 13 | 0.30 | 0.30 | 0.30 | 0.30 | 0.30 | 0.30 |
| PHENOXYETHANOL | 14 | 0.50 | 0.50 | 0.50 | 0.50 | 0.50 | 0.50 |
| TOTAL | | 100.00 | 100.00 | 100.00 | 100.00 | 100.00 | 100.00 |

| | | | |
|---|---|---|---|
| BUBBLES | NO | YES (+) | YES (++) |

(1) DOW CORNING VM-2270 AEROGEL, 98% AM; Specific surface = 600-800 m²/g
(2) CABOT CAB-O-SIL TS-530, 100% AM; Specific surface = 205-245 m²/g

Bubbles were evaluated by applying an equal amount of Part A and Part B formulas on a glass slide and mixed, before covering with a cover slip. Bubble formation was observed within seconds.

Table 2 demonstrates that at vinyl-terminated polysiloxane to hydride-functionalized polysiloxane molar ratio above 1:25, there was formation of bubbles upon mixing Part A and Part B of the formulas (Ex. 2C-1A with 2C-1B, or 2C-2A with 2C-2B), while the mixture of Ex. 2-1A with 2-1B, at vinyl-terminated polysiloxane to hydride-functionalized polysiloxane molar ratio of 1:3, there were no bubbles.

### Example 3: Application of Composition

A two-step application requires the user to first apply the Part A product and then layer on top the Part B product. For a one-step application process, both parts may be contained in a double chamber pack and dispenses simultaneously on a spatula, and mixed directly on the skin to form a homogeneous film. Thus, a one-step application mode may be preferred for better practical compliance.

A one-step application may require to have the same viscosity of parts (A) and (B), and thus to have the same content of vinyl-terminated polysiloxane and fillers in part (A) and (B), or the film may be rendered inhomogeneous.

The compositions for Ex. 3-1B and 3-2B were prepared as follows. Components 3-10 were mixed and homogenized at 500 rpm. In a separate container, components 13-20 were stirred for 10 minutes at 72° C. (Mixture B). Mixture B was added slowly to mixture A under strong agitation (500 rpm). The mixing speed was increased progressively while adding mixture B, to reach 1050 rpm at the end. The formula was finalized for 10 minutes at 1350 rpm and confirmed as homogeneous.

The remaining compositions were prepared as follows. Components 1-6 were mixed for 3 minutes at 2500 rpm. After pre-melting component 11, components 9-12 were added to the mix, and mixed for 2 minutes at 2500 rpm (Mixture A). In a separate container, components 13-20 were stirred for 10 minutes at 72° C. (Mixture B). Mixture B was added to mixture A under strong agitation (700 rpm). The mixing speed was increased progressively while adding mixture B, to reach 1000 rpm at the end. The formula was finalized for 10 minutes at 1100 rpm and confirmed as homogeneous.

TABLE 3A
Evaluation of one-step vs. two-step process

| | | 2 steps | | 1 step | | 1 step | |
|---|---|---|---|---|---|---|---|
| | | Ex. 3-1 | | Ex. 3-2 | | Ex. 3-3 | |
| INCI name | Component # | 3-1A | 3-1B | 3-2A | 3-2B | 3-3A | 3-3B |
| VINYL-TERMINATED PDMS | 1 | 20 | | 20 | | 20 | 20 |
| POLYDIMETHYL-METHYLHYDROSILOXANE | 2 | 0.4 | | 0.4 | | 0.4 | |
| PLATINUM- | 3 | | 1 | | 1 | | 0.7 |
| DIVINYLTETRAMETHYLDISILOXANE | | | | | | | |
| COMPLEX | | | | | | | |
| TRISILOXANE | 4 | 38.6 | 20 | 38.6 | 20 | 38.6 | 38.3 |
| SILICA SILYLATE (1) | 5 | 2.5 | | 2.5 | | 2.5 | 2.5 |
| SILICA SILYLATE (2) | 6 | 1.67 | | 1.67 | | 1.67 | 1.67 |
| NYLON-12 | 7 | | 5 | | 5 | | |
| DIMETHICONE (and) DIMETHICONE | 8 | | 2 | | 2 | | |
| CROSSPOLYMER | | | | | | | |
| DIMETHICONE (and) DIMETHICONE/PEG- | 9 | 2.17 | 1 | 2.17 | 1 | 2.17 | 2.17 |
| 10/15 CROSSPOLYMER | | | | | | | |
| DIMETHICONE (and) PEG/PPG-18/18 | 10 | 0.57 | 1.8 | 0.57 | 1.8 | 0.57 | 0.57 |
| DIMETHICONE | | | | | | | |
| PEG-30 DIPOLYHYDROXYSTEREATE | 11 | 0.54 | | 0.54 | | 0.54 | 0.54 |
| PEG-10 DIMETHICONE | 12 | 0.72 | | 0.72 | | 0.72 | 0.72 |
| WATER | 13 | 31.03 | 36.41 | 31.03 | 36.41 | 31.03 | 31.03 |
| BUTYLENE GLYCOL | 14 | | 9 | | 9 | | |
| PROPYLENE GLYCOL | 15 | | 18 | | 18 | | |
| GLYCERIN | 16 | | 4 | | 4 | | |
| MAGNESIUM SULFATE | 17 | 1 | | 1 | | 1 | 1 |
| SODIUM CHLORIDE | 18 | | 0.99 | | 0.99 | | |
| CAPRYLYL GLYCOL | 19 | 0.3 | 0.3 | 0.3 | 0.3 | 0.3 | 0.3 |
| PHENOXYETHANOL | 20 | 0.5 | 0.5 | 0.5 | 0.5 | 0.5 | 0.5 |
| TOTAL | | 100 | 100 | 100 | 100 | 100 | 100 |
| VISCOSITY (at 118 1/s) | | 2.6 | 1.1 | 2.6 | 1.1 | 2.6 | 2.8 |
| | | Pa · s | Pa · s | Pa · s | Pa · s | Pa · s | Pa · s |

| | | | |
|---|---|---|---|
| APPLICATION | 2 steps | 1 step | 1 step |
| FILM QUALITY | Good | Inhomogeneous | Homogeneous |

Table 3 shows that when the Part A and B of the formulas have different viscosities (Ex. 3-2A and 3-2B), they do not mix well, and thus lead to an inhomogeneous film. If applied in 2 steps (Part A first then part B on top of Part A), then the film is homogenous. We also showed that if Part A and Part B of the formula have the same viscosity (Ex. 3-3A and 3-3B), then the two parts mix well and the film is homogenous.

### Example 4: Panel/Sensory Testing

The compositions were tested on a panel of 6 people of grade 4 eye bags (based on Atlas' score from 0 to 5) and showed stronger and longer performance (up to 6 hours) on eye bags, under eye wrinkles, crow's feet, and glabellar lines than a comparative example.

The compositions for Ex. 4-1A and 4-1B were prepared as follows. Components 1-8 were mixed for 3 minutes at 2500 rpm. After pre-melting component 14, components 12-15 were added to the mix, and mixed for 2 minutes at 2500 rpm (Mixture A). In a separate container, components 20-28 were stirred for 10 minutes at 72° C. (Mixture B). Mixture B was added to mixture A under strong agitation (700 rpm). The mixing speed was increased progressively while adding mixture B, to reach 1000 rpm at the end. The formula was finalized for 10 minutes at 1100 rpm and confirmed as homogeneous.

The compositions for Ex. 4C-1A and 4C-1B were prepared as follows. Components 1-9 were mixed for 5 minutes at 2500 rpm (Mixture A). Components 16-19 were added to mixture A and mixed for 5 minutes at 2500 rpm. In a separate container, components 20-28 were stirred for 5 minutes (Mixture B). Mixture B was added slowly to mixture A under strong agitation (500 rpm for 10 minutes) to make Mixture C. Component 10 was added in mixture C at the end and mixed in for 5 minutes at 1000 rpm. Components 5-13 were mixed and homogenized at 500 rpm. In a separate container, components 20-27 were stirred for 10 minutes at 72° C. (Mixture B). Mixture B was added slowly to mixture A under strong agitation (500 rpm). The mixing speed was increased progressively while adding mixture B, to reach 1050 rpm at the end. The formula was finalized for 10 minutes at 1350 rpm and confirmed as homogeneous.

TABLE 4A
Panel testing-compositions

| | | Ex. 4-1 | | Ex. 4C-1 | |
|---|---|---|---|---|---|
| INCI name | Component # | 4-1A | 4-1B | 4C-1A | 4C-1B |
| VINYL-TERMINATED PDMS (5) | 1 | 20.00 | 20.00 | | |
| VINYL-TERMINATED PDMS (6) | 2 | | | 23.80 | |
| VINYL-TERMINATED PDMS (7) | 3 | | | 5.55 | |
| POLYDIMETHYL-METHYLHYDROSILOXANE | 4 | 0.40 | | 12.00 | |
| PLATINUM-DIVINYLTETRAMETHYLDISILOXANE | 5 | | 0.70 | | 1.00 |
| COMPLEX | | | | | |
| TRISILOXANE | 6 | 38.60 | 38.30 | | 20.00 |
| SILICA SILYLATE (1) | 7 | 2.50 | 2.50 | | |
| SILICA SILYLATE (2) | 8 | 1.67 | 1.67 | | |
| SILICA SILYLATE (3) | 9 | | | 9.45 | |
| NYLON-12 | 10 | | | 5.90 | 5.00 |
| DIMETHICONE (and) DIMETHICONE | 11 | | | | 2.00 |
| CROSSPOLYMER | | | | | |
| DIMETHICONE (and) DIMETHICONE/PEG-10/15 | 12 | 2.17 | 2.17 | | 1.00 |
| CROSSPOLYMER | | | | | |
| DIMETHICONE (and) PEG/PPG-18/18 | 13 | 0.57 | 0.57 | | 1.80 |
| DIMETHICONE | | | | | |
| PEG-30 DIPOLYHYDROXYSTEREATE | 14 | 0.54 | 0.54 | | |
| PEG-10 DIMETHICONE | 15 | 0.72 | 0.72 | | |
| C30-45 ALKYL CETEARYL DIMETHICONE | 16 | | | 3.60 | |
| CROSSPOLYMER | | | | | |
| POLYSILICONE-11 (and) LAURETH-6 | 17 | | | 3.60 | |
| METHYL METHACRYLATE CROSSPOLYMER | 18 | | | 1.20 | |
| POLYACRYLATE-13 (and) POLYISOBUTENE (and) | 19 | | | 1.20 | |
| POLYSORBATE 20 | | | | | |
| WATER | 20 | 31.03 | 31.03 | 27.00 | 36.41 |
| BUTYLENE GLYCOL | 21 | | | | 9.00 |
| PROPYLENE GLYCOL | 22 | | | | 18.00 |
| GLYCERIN | 23 | | | | 4.00 |
| MAGNESIUM SULFATE | 24 | 1.00 | 1.00 | | |
| SODIUM CHLORIDE | 25 | | | | 0.99 |
| CAPRYLYL GLYCOL | 26 | 0.30 | 0.30 | | 0.30 |
| PHENOXYETHANOL | 27 | 0.50 | 0.50 | | 0.50 |
| BUTYLENE GLYCOL (and) GLYCERYL | 28 | | | 6.70 | |
| POLYACRYLATE (and) PVM/MA COPOLYMER | | | | | |

| | | | | |
|---|---|---|---|---|
| TOTAL | 100.00 | 100.00 | 100.00 | 100.00 |

The panel testing protocol was as follows. Panelists cleaned there face with a cleanser, and re-equilibrated for 10 minutes. An expert applied a moisturizer and waited 2 minutes before applying the invention or comparative product on the under eye area to cover eye bags and under eye wrinkles, the crow's feet, and the glabellar lines. Images were taken, and panelists were evaluated by the expert at: Baseline, 10 minutes, 30 minutes, 3 hours and 6 hours. The expert graded the attributes on a scale from 0 to 5 (none to severe). The results are shown in Tables 4B-1 and 4B-2.

TABLE 4B-1
Panel testing-results (Ex. 4-1)

| GRADE LOSS AT | BASELINE | 10 min | 3 hrs | 6 hrs |
|---|---|---|---|---|
| Eye bags | 0 | −1 | −1.5 | −1.3 |
| Under eye wrinkles | 0 | −2.2 | −2 | −1.8 |
| Crow's feet | 0 | −0.8 | −1.3 | −1 |
| Glabellar lines | 0 | −0.3 | −0.8 | −0.3 |

TABLE 4B-2
Panel testing-results (Ex. 4C-1)

| GRADE LOSS AT | BASELINE | 10 min | 3 hrs | 6 hrs |
|---|---|---|---|---|
| Eye bags | 0 | −0.3 | −1.3 | −0.7 |
| Under eye wrinkles | 0 | −0.5 | −1 | −0.7 |
| Crow's feet | 0 | −0.2 | −0.8 | 0 |
| Glabellar lines | 0 | −0.3 | −0.3 | 0 |

The results demonstrate that Ex. 4-1 decreased the appearance of eye bags, under eye wrinkles, crow's feet and glabellar lines more than the comparative examples, at all time points (10 minutes, 3 hours, and 6 hours) relative to Ex. 4C-1. With the composition of Ex. 4-1, the grade loss for eye bags was between 1 and 1.5, and for under eye wrinkles was between 1.8 and 2.2 on a scale of 5. With the comparative examples, the grade loss for eye bags was between 0.3 and 1.3, and for under eye wrinkles was between 0.5 and 1 on a scale of 5. It is further noted that Ex. 4-1 performance lasted longer as the appearance of all attributes were still reduced at 6 hours compared to baseline, while the appearance of eye bags and under eye wrinkles only were still reduced at 6 hours with the comparative product.

We claim:

1. A skin tightening composition comprising:
(A) (a) at least one vinyl-terminated polysiloxane and at least one hydride-functionalized polysiloxane;
(b) at least one filler; and
(B) (a) at least one vinyl-terminated polysiloxane;
(c) at least one filler; and
(d) at least one at least one metal catalyst,
wherein the viscosity of the at least one vinyl-terminated polysiloxane ranges from about 50,000 to about 100,000 cSt at 25° C., and
wherein the specific surface area of the at least one filler is greater than about 300 m²/g.

2. The skin tightening composition of claim 1, wherein the molar ratio of the at least one vinyl-terminated polysiloxane and at least one hydride-functionalized polysiloxane ranges from about 1:1 to about 1:20.

3. The skin tightening composition of claim 1, wherein the total amount of reactive components according to various embodiments of the disclosure ranges from about 10% to about 65% by weight, relative to the total weight of the composition.

4. The skin tightening composition of claim 1, wherein the at least one vinyl-terminated polysiloxane is chosen from compounds of formula I: 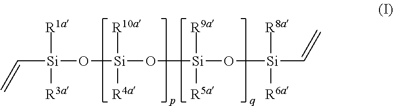
wherein:
each of R^{1a′}, R^{3a′}, R^{4a′}, R^{5a′}, R^{6a′}, R^{8a′}, R^{9a′}, and R^{10a′} are independently chosen from hydrogen, C₁-C₂₀ alkyl, C₂-C₂₀ alkenyl, C₅-C₁₀ aryl, hydroxyl, or C₁-C₂₀ alkoxyl; and
p and q are integers independently ranging from about 10 to about 6000.

5. The skin tightening composition of claim 4, wherein the at least one vinyl-terminated polysiloxane is chosen from vinyl terminated polydimethylsiloxane; vinyl terminated diphenylsiloxane-dimethylsiloxane copolymers; vinyl terminated polyphenylmethylsiloxane, vinylphenylmethyl terminated vinylphenylsiloxane-phenylmethylsiloxane copolymer; vinyl terminated trifluoropropylmethylsiloxane-dimethylsiloxane copolymer; vinyl terminated diethylsiloxane-dimethylsiloxane copolymer: vinylmethylsiloxane-dimethylsiloxane copolymer, trimethylsiloxy terminated; vinylmethylsiloxane-dimethylsiloxane copolymers, silanol terminated; vinylmethylsiloxane-dimethylsiloxane copolymers, vinyl terminated; vinyl gums; vinylmethylsiloxane homopolymers; vinyl T-structure polymers; monovinyl terminated polydimethylsiloxanes; vinylmethylsiloxane terpolymers; vinylmethoxysilane homopolymers, and combinations thereof.

6. The skin tightening film of claim 1, wherein the at least one vinyl-terminated polysiloxane is present in the composition in an amount ranging from about 10% to about 60%, by weight, relative to the total weight of the composition.

7. The skin tightening film of claim 1, wherein the at least one vinyl-terminated polysiloxane is present in each of parts (A) and (B), independently, of the composition in an amount ranging from about 10% to about 30%, by weight, relative to the total weight of the part in which it is present.

8. The skin tightening composition of claim 1, wherein the at least one hydride-functionalized polysiloxane has a viscosity ranging up to about 100 cSt at 25° C.

9. The skin tightening composition of claim 8, wherein the at least one hydride-functionalized polysiloxane is chosen from compounds of formula (II): 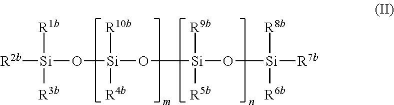
wherein:
R^{1b}, R^{2b}, R^{3b}, R^{4b}, R^{5b}, R^{6b}, R^{7b}, R^{8b}, R^{9b} and R^{10b} are each independently chosen from hydrogen, C₁₋₂₀ alkyl, C₂₋₂₀ alkenyl, C₅₋₁₀ aryl, hydroxyl or C₁₋₂₀ alkoxy, and
m and n are each independently an integer ranging from about 10 to about 6000,
with the proviso that at least one of R^{1b}, R^{2b}, R^{3b}, R^{4b}, R^{5b}, R^{6b}, R^{7b}, R^{8b}, R^{9b} and R^{10b} is hydrogen.

10. The skin tightening composition of claim 9, wherein the at least one hydride-functionalized polysiloxane is chosen from hydride terminated polydimethylsiloxane; polyphenyl-(dimethylhydrosiloxy)siloxane, hydride terminated; methylhydrosiloxane-phenylmethylsiloxane copolymer, hydride terminated; methylhydrosiloxane-dimethylsiloxane copolymers, trimethylsiloxy terminated; polymethylhydrosiloxanes, trimethylsiloxy terminated; polyethylhydrosiloxane, triethylsiloxane, methylhydrosiloxane-phenyloctylmethylsiloxane copolymer; methylhydrosiloxane-phenyloctylmethylsiloxane terpolymer, and combinations thereof.

11. The skin tightening composition of claim 1, wherein the at least one hydride-functionalized polysiloxane is present in the composition in an amount ranging from about 0.01% to about 3% by weight, relative to the weight of the composition.

12. The skin tightening composition of claim 1, wherein the at least one filler is chosen from silica.

13. The skin tightening composition of claim 12, wherein the at least one filler is chosen from hydrophobic silica aerogel particles.

14. The skin tightening composition of claim 12, wherein the at least one filler is present in the composition in a total amount ranging from up to about 8% by weight, relative to the total weight of the composition.

15. The skin tightening composition of claim 1, wherein the at least one metal catalyst is chosen from platinum catalysts.

16. The skin tightening composition of claim 1, further comprising at least one additional components chosen from volatile solvents, emulsifiers, colorants, silicone elastomers, and humectants.

17. The skin tightening composition of claim 16, wherein the at least one solvent is chosen from volatile solvents.

18. The skin tightening composition of claim 17, wherein the at least one solvent is chosen from volatile hydrocarbon-based oils and volatile silicone oils.

19. A skin tightening film formed from the composition of claim 1, wherein the film has a Young Modulus greater than about 500 kPa.

20. The skin tightening film of claim 19, wherein the Young Modulus of the film is greater than about 1000 kPa.

21. The skin tightening film of claim 20, wherein the Young Modulus of the film is greater than about 5000 kPa.

22. A method for improving the appearance of the skin, said method comprising forming a film on the skin by applying to the skin a composition comprising:
(A) (a) at least one vinyl-terminated polysiloxane and at least one hydride-functionalized polysiloxane;
(b) at least one filler; and
(B) (a) at least one vinyl-terminated polysiloxane;
(b) at least one filler; and
(c) at least one at least one metal catalyst,
wherein the viscosity of the at least one vinyl-terminated polysiloxane ranges from about 50,000 to about 100,000 cSt at 25° C., and
wherein the specific surface area of the at least one filler is greater than about 300 m²/g.

23. The method of claim 22, wherein the Young Modulus of the film ranges from about 500 kPa to about 2000 kPa.

24. The method of claim 22, wherein the Young Modulus of the film ranges from about 500 kPa to about 50,000 kPa.

25. A kit comprising a composition comprising:
(A) (a) at least one vinyl-terminated polysiloxane and at least one hydride-functionalized polysiloxane;
(b) at least one filler; and
(B) (a) at least one vinyl-terminated polysiloxane;
(b) at least one filler; and
(c) at least one at least one metal catalyst,
wherein the viscosity of the at least one vinyl-terminated polysiloxane ranges from about 50,000 to about 100,000 cSt at 25° C., and
wherein the specific surface area of the at least one filler is greater than about 300 m²/g;
wherein parts (A) and (B) are packaged in separate compartments.
